(19) United States
(12) Patent Application Publication
(10) Pub. No.: US 2026/0157662 A1
(43) Pub. Date: Jun. 11, 2026

(54) DEVICE FOR DYNAMIC DETERMINATION OF A BASAL INSULIN DOSE TO BE INJECTED

(71) Applicant: DIAPPYMED, CLAPIERS (FR)

(72) Inventors: Omar DIOURI, CLAPIERS (FR); Youssef RAQUI, CLAPIERS (FR)

(21) Appl. No.: 18/706,013

(22) PCT Filed: Nov. 18, 2022
(86) PCT No.: PCT/EP2022/082464

§ 371 Date: Apr. 30, 2024

(30) Foreign Application Priority Data
Nov. 18, 2021 (FR) FR2112226

(51) Int. Cl. A61B 5/145; A61B 5/00

(52) U.S. Cl. CPC A61B 5/14532; A61B 5/7282; A61B 5/742; A61B 5/746; A61B 5/7475

(57) ABSTRACT

The device for dynamic determination of a slow insulin dose to be injected comprises:
a means (21) of acquiring multiple blood glucose levels during a first day and the day following the first day,
a means (26) of calculating the difference in blood glucose levels,
a means (22, 24, 25, 42, 43) of determining if a predetermined event has occurred between going to bed and getting up,
a means (26) of determining, if no predetermined event has occurred, a hyperglycaemia or a hypoglycaemia depending on the difference in blood glucose levels,
a means (26) of determining if at least a predetermined number of hyperglycaemias or hypoglycaemias have occurred in a predetermined period of time,
a means (26) of modifying the slow insulin to be injected, in this case of an occurrence, and
a means (25) of indicating a numerical value of the modified slow insulin dose to be injected.

### TECHNICAL FIELD OF THE INVENTION

The present invention relates to a device for the dynamic determination of a basal insulin dose to be injected in the context of treatment with multiple injections, and more specifically for a user with type 1 diabetes. It applies, in particular, to the field of medical devices used by the patient.

### STATE OF THE ART

Type 1 diabetes (“T1D”) affects about 350,000 people in France, and almost six million people in Europe. For 20 years, the number of people with type 1 diabetes (“PT1D”) has increased by 3-4% each year. The causes of this rise are as yet unknown, but as the factors involved are environmental, genetic and nutritional, this increase is expected to continue and intensify.

The bodies of patients with type 1 diabetes do not produce insulin (essential for converting the carbohydrates in meals into energy, and thus regulating the level of glucose in the blood). These patients must therefore administer insulin several times a day. This involves:

A continuous control of the glycaemia (blood glucose level) by a device for measuring the blood glucose, called a glucometer, dextro, or glucose sensor.

A minimum of four insulin injections are needed each day, using a device to administer the insulin, called an insulin pen. These injections consist of basal insulin, also called slow insulin (background insulin, to be administered once a day), and fast-acting insulin (to be administered before each food intake, called bolus). These injections also include correction operations (re-injecting a low dose in the event of a hyperglycaemia).

The aim of these different steps is to maintain the patients' blood glucose level within a range of values of 70-180 mg/dL. When the blood glucose level is below this value range, this indicates a hypoglycaemia, which can cause, in advanced cases, unconsciousness and coma. When the blood glucose level is above this value range, this indicates a hyperglycaemia, which can cause, in the long term, diabetic imbalance.

Diabetes imbalance causes events that are harmful for the organs, and long-term serious complications such as atherosclerosis, myocardial infarction, stroke and arteritis of the lower limbs, and also retinopathies and nephropathies. Indeed diabetes increases the risk of amputation by a factor of eight, and the risk of dialysis because of renal failure by a factor of nine. It is the second leading cause of cardio-vascular incidents, and the leading cause of blindness in adults.

Generally speaking, slow insulin, or basal insulin, makes it possible to meet the body's minimum insulin requirements, in the absence of carbohydrate intakes. In the case of people using insulin pump therapy, this insulin is perfused continuously in the body and the rate is expressed in U/h. This rate can be fine-tuned for each time period. On the other hand, in the case of people treated by multiple injections, this insulin is taken in the form of a daily injection. Such a single dose must be adjusted to cover the requirements for the whole day. Note that a basal insulin level that is:
too high results in an increase in day-time and night-time hypoglycaemias; and
too low results in a rise in the blood glucose level.

Many people with type 1 diabetes have a poorly-controlled treatment. For example, the SAGE study presented at the EASD annual meeting in 2019, carried out in 17 countries in Western Europe, Eastern Europe, Latin America, Asia and the Middle East, showed that more than 40% of patients had an Hb1Ac level (Glycated haemoglobin, which reflects the average blood glucose levels of the last three months) higher than 8%, reflecting a poorly controlled diabetes. In addition, 43% of patients did not adjust their insulin doses themselves, since they were dependent on the doctor or medical team.

Functional insulin therapy makes it possible to control the diabetes, but is not very accessible. It has been shown that adjusting the dose enables the HbA1c level to be reduced by 1%. This reduction can, in many cases, make it possible to keep diabetes “managed”. In addition, studies have already demonstrated that this would enable a 30% reduction in the long-term complications.

Functional insulin therapy consists of calculating the optimum insulin dose to be administered, based on the foods ingested and other physiological parameters, instead of taking a fixed dose. Currently, this adjustment is difficult to implement. Patients who want to use this method must receive special training, acquire a lot of knowledge and then, at each meal, make complicated calculations perceived as risky and time-consuming. These calculations are then applied to each injection. Only 20% of patients can undertake this approach, and some of them make the calculations by incorporating personal simplifications and shortcuts, risking lower efficiency and therefore unbalanced diabetes.

Decision-support systems for people with type 1 diabetes that are currently available on the market only offer a standard bolus calculator, requiring prior knowledge of the calculation parameters and mastery of carbohydrate counting and functional insulin therapy by the user, reducing the number of people able to use this method. Nor do these systems offer a dynamic adjustment of the slow insulin to be injected, and the few devices handling slow insulin are only designed for people with type 2 diabetes not making multiple injections.

In addition, it is noted that, because type 1 diabetes and type 2 diabetes differ in their cause, and therefore physiopathology, the treatments associated with these two types of diabetes are different:
people with type 1 diabetes are usually treated with multiple injections of slow and/or fast-acting insulin, the slow insulin being injected once a day and the fast-acting insulin being injected before each meal; and
people with type 2 insulin are usually treated by administering oral anti-diabetics.

Thus, the respective therapies for people with type 1 or type 2 diabetes are fundamentally different.

European patent EP 285 94 80 is known, which discloses a device for managing the perfusion of insulin. In particular, such an insulin perfusion is controlled by an insulin pump system. An insulin pump provides, among other things, a basal flow rate given in U/hour, and users of the management device comprising such a pump have algorithms for controlling the basal flow rate dynamically.

However, the adjustment of the insulin administered by such a device is made on the basis of a time band, not incrementally as implemented by an insulin pen. Therefore, such a device is not suitable for the administration of insulin according to a protocol of multiple injections taken just once a day and administered by a pen.

In addition, the insulin perfusion management device is a decision support tool for the doctor, such a decision consisting of choosing the pump flow rate to be applied. Amongst others, such a device displays a report for the doctor indicating the need to reduce or increase the flow rate of insulin injected, without defining a value. In other words, no precise data is indicated to the doctor, and it is therefore up to the doctor to determine the adjustment value of the flow rate of insulin to be injected. Such an adjustment is therefore made on an ad-hoc basis by the doctor, and only when the device displays a report.

International patent application WO 2020/074500 is also known, which discloses a device helping to direct the drug therapy of a diabetic patient. It is noted that this device is particularly suitable for oral anti-diabetics and for patients with type 2 diabetes. In addition, this device makes between 4 and 6 ad-hoc measurements during the day, and their assignment to an event in the day is made directly by the user. In particular, such a device uses, for example, data on capillary blood glucose, known as “Self Monitoring Blood Glucose”, acronym “SMBG”, for which the associated values have already been assigned.

The devices of the prior art are therefore contrary to a functional insulin therapy intended to improve the autonomy of patients. In addition, it is noted that the vast majority of patients use insulin pens and therefore do not benefit from a dynamic adjustment system suited to their insulin injection system.

### Presentation of the Invention

The present invention aims to remedy all or part of these drawbacks.

To this end, according to a first aspect, the present invention envisions a device according to claim 1 for dynamically determining a slow insulin dose to be injected for a user treated by multiple injections.

Thanks to the utilisation of the present invention, the slow insulin dosage is better adjusted and more accurate for the user treated by multiple injections, i.e. who injects several doses of insulin each day and uses a Continuous Glucose Monitor, acronym “CGM”, compared to methods using only capillary fasting glucose tests (self-monitoring blood glucose, acronym “SMBG”). In addition, thanks to these provisions, the indication, labelling or tagging of the blood sugar level no longer has to be performed by the user. In other words, the overall automation of the device releases the user from indicating the nature of the blood sugar level acquired.

Thus, such a device makes it possible to implement a dynamic, automated functional insulin therapy for the user treated by multiple injections. The patient's therapeutic autonomy is therefore improved.

When the predefined period is, for example, seven days, therefore each week, if no predefined event has occurred between going to bed and getting up, or fasting state, the device recommends:
increasing or reducing the slow insulin dose to be injected, if the number of hypoglycaemias or hyperglycaemias that occurred during these seven days is greater than or equal to two; or
maintaining the slow insulin dose to be injected, if the number of hypoglycaemias or hyperglycaemias that occurred during these seven days is less than or equal to two. In this case, the device takes into account all the data of the seven-day week, in particular all the blood sugar differences.
   On the other hand, if a predefined event occurs between the user going to bed and getting up, or fasting state, the device does not take into account the blood sugar differences where these events occur. In addition, it is noted that the device indicates the slow insulin dose to be injected in the form of a numerical value.

In some optional embodiments, the device also comprises:
a means for detecting an increase in the numerical value of the modified slow insulin dose to be injected;
a determination means, activated by the detection means, for determining whether at least a predetermined number of hypoglycaemias greater than or equal to one has occurred in a second predetermined period of time, the second period of time being less than the first period of time; and
a correction means for correcting the numerical value of the slow insulin dose to be injected if at least the predetermined number of hypoglycaemias has occurred in the second period of time, the correction means being configured to transmit to the indication means a corrected numerical value of the slow insulin dose to be injected.

Thanks to these provisions, the device enables:
greater monitoring of the occurrence of a hypoglycaemia during an increase in the slow insulin dose to be injected; and
a correction of the slow insulin dose, if necessary.

In this way, the risks of repeated hypoglycaemia after the injection of the slow insulin dose are limited.

In some optional embodiments, the means for determining if a predetermined event has occurred between this going to bed and this getting up is configured to determine the occurrence of at least one event from amongst:
having a meal between going to bed and getting up, or fasting state;
carrying out a physical activity between going to bed and getting up, or fasting state; and
taking insulin between going to bed and getting up, or fasting state.

The applicant has determined that these events can affect the blood glucose level and that their occurrence must inhibit the difference in blood glucose levels being taken into account for the night considered.

In some optional embodiments, the device also comprises:
a means for determining the occurrence of an event from amongst an illness of the user, the user's menstruation, and carrying out a physical activity;
a means for determining whether at least a predetermined number, greater than or equal to one, of hypoglycaemias or hyperglycaemias has occurred in a predetermined period of time specific to this event, the means for determining the number of hypoglycaemias or hyperglycaemias being activated by the occurrence of a said event; and
a means for modifying the slow insulin dose to be injected by a specific slow insulin dose, if at least the predetermined number of hypoglycaemias or hyperglycaemias has occurred in the predetermined specific period of time,
the indication means indicating to the user a numerical value of the specific slow insulin dose to be injected if the occurrence of a said event is determined.

Thanks to these provisions, the device enables a specific adjustment of the slow insulin dose to be injected based on the presence of a particular state, illness, menstruation or physical activity, of the user. Thus, the device can be personalised according to the user.

In some optional embodiments, the device also comprises a means for determining a correction bolus or a meal bolus, based on:
the last blood glucose level measured in the user's blood;
a correction factor (CF);
an upper target blood glucose level; and
a lower target blood glucose level,
if the occurrence of an event, from amongst an illness of the user, the user's menstruation, and carrying out a physical activity, is determined.

In some optional embodiments, the device also comprises a means for determining whether the correction bolus is higher than a minimum injectable fast-acting insulin dose; and:
if it is, transmitting to the user the value of the correction bolus to be injected; and
if not, transmitting to the user a value representative of a physical activity making it possible to obtain the same effect as the corrective insulin dose.

In some optional embodiments, the device also comprises:
a means for determining a blood glucose level of the user before a meal, called the “pre-prandial blood glucose”, the pre-prandial blood glucose being labelled by the assignment means;
a means for determining a blood glucose level of the user after a meal, called the “post-prandial blood glucose”, the post-prandial blood glucose being labelled by the assignment means;
a means for receiving a description of a meal;
a means for determining an insulin active at the time of the injection;
a means for determining a new glucose ratio for a meal timeslot between the acquisition instants labelled as pre-prandial blood glucose level and those labelled as post-prandial blood glucose level, as a function of the following factors:
a pre-prandial blood glucose level;
a post-prandial blood glucose level;
an amount of carbohydrates absorbed during the meal;
a bolus;
a level of active insulin;
a value of a correction factor;
an upper target blood glucose level; and
a lower target blood glucose level; and
a means for replacing a glucose ratio used by the device for determining a fast-acting insulin dose to be injected, representative of a number of carbohydrates absorbed by the user, if at least a predetermined number, greater than or equal to two, of new glucose ratios were determined in a predetermined period of time, the replacement glucose ratio being an average, possibly weighted, of the new glucose ratios.

In some optional embodiments, the determining means determines a new specific glucose ratio for a meal timeslot as a function, additionally, of the following factors:
an illness of the user;
the user's menstruation; and/or
carrying out a physical activity,
the device also comprising a means for replacing a glucose ratio by a specific glucose ratio used by the device for determining a specific fast-acting insulin dose to be injected, the replacement specific glucose ratio being determined as a function of the presence of an illness and/or menstruation.

In some optional embodiments, the means for replacing a glucose ratio is configured to inhibit the replacement of the glucose ratio if the replacement glucose ratio is within a predefined range of values around the glucose ratio to be replaced.

In some optional embodiments, the device also comprises:
a means for receiving a description of a meal;
a means for calculating nutriments corresponding to the meal described, number of calories, carbohydrates, lipids and proteins;
a means for determining a correlation between the presence of the foods described and a tendency to hypoglycaemia or hyperglycaemia after meals; and
a means for triggering an alert for the user, if a sufficiently high correlation is detected; and
a means for determining a meal bolus dose, as a function of the glucose ratio (ICR) and correction factor (CF) values.

In some optional embodiments, the device also comprises a means for the daily determination of a new correction factor (CF) corresponding to a factor lowering blood glucose levels for a unit of insulin received, the correction factor being a function of the following factors:
a pre-correction blood glucose level;
a post-correction blood glucose level;
a bolus;
an active insulin;
an upper target blood glucose level; and
a lower target blood glucose level; and
a means for replacing a correction factor for determining a fast-acting insulin dose to be injected, if at least a predefined number, greater than or equal to two, of new correction factors were determined in a predetermined period of time, the replacement correction factor being an average, possibly weighted, of the new correction factors.

In some optional embodiments, the daily determining means determines a new specific correction factor as a function, additionally, of the following factors:
an illness of the user; and/or
the user's menstruation,
the device also comprising a means for replacing a correction factor by a specific correction factor for determining a specific fast-acting insulin dose to be injected, the replacement specific correction factor being determined as a function of the presence of an illness and/or menstruation.

In some optional embodiments, the means for replacing a correction factor is configured to inhibit the replacement of the correction factor if the replacement correction factor is within a predetermined range of values around the correction factor to be replaced.

According to a second aspect, the present invention envisions a method according to claim 14 for dynamically determining a slow insulin dose to be injected for a user treated by multiple injections.

As the particular features, advantages and aims of this method are similar to those of the device that is the subject of the invention, they are not repeated here.

### BRIEF DESCRIPTION OF THE FIGURES

Other advantages, aims and particular features of the invention will become apparent from the non-limiting description that follows of at least one particular embodiment of the device and method for dynamically determining a slow insulin dose to be injected that are the subjects of the present invention, with reference to drawings included in an appendix, wherein:

FIG. 1 represents, schematically, a particular embodiment of a device that is the subject of the invention;

FIG. 2 represents, in the form of a logic diagram, steps in calculating a glucose ratio, or “ICR”;

FIG. 3 represents, in the form of a logic diagram, steps in calculating a specific glucose ratio, or specific “ICR”;

FIG. 4 represents, in the form of a logic diagram, steps in calculating a correction factor “CF”;

FIG. 5 represents, in the form of a logic diagram, steps in calculating a specific correction factor “CF”;

FIG. 6 represents, in the form of a logic diagram, steps in calculating a slow insulin;

FIG. 7 represents, in the form of a logic diagram, steps in calculating a specific slow insulin;

FIG. 8 represents, in the form of a logic diagram, steps in calculating a meal bolus;

FIG. 9 represents, in the form of a logic diagram, steps in calculating a correction bolus;

FIG. 10 represents, in the form of a logic diagram, steps in determining slow insulin; and

FIG. 11 represents the happenings in a day and the blood glucose measurements utilised.

### DESCRIPTION OF THE EMBODIMENTS

The present description is given in a non-limiting way, in which each characteristic of an embodiment can be combined with any other characteristic of any other embodiment in an advantageous way.

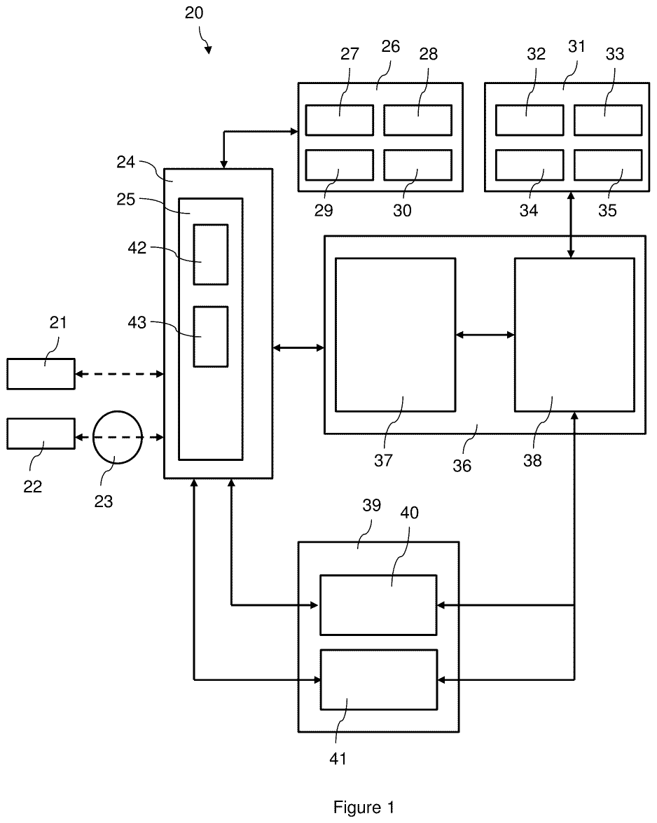

First of all, the functional components of an embodiment of the device that is the subject of this invention are described below, with respect to FIG. 1. In some embodiments, the device 20 is installed manually on the user's arm, by a doctor or a nurse, for example. In particular, after the device 20 has been installed on the user, such a device 20 performs automatic operations, i.e. without human intervention and therefore non-manual. Such operations correspond, for example, to acquiring blood glucose data or assigning these data to times of at least one day.

This device 20 comprises a user interface 25, preferably on a communicating terminal 24 of the user, for example a computer, tablet or mobile telephone and, more preferably, a smartphone.

This user interface 25 comprises, preferably, a keyboard 42, possibly in the form of a touch screen and a speech recognition module 43, utilising a microphone of the user terminal 24.

The user terminal 24 utilises means for communicating with a blood glucose meter 21, for example utilising the Bluetooth (registered trademark) communication protocol, with a connected watch 22, for example equipped with an actimeter, and/or with a connected insulin pen, for example by means of a server 23 of the provider of this connected watch or this connected insulin pen.

The terminal 24 verifies that the inputs and outputs are correct.

A calculator 26, for example on a web server, comprises a module 27 for calculating 10B (acronym for “Insulin on board”, also called “active insulin”). 10B refers to the amount of insulin from the bolus that was recently administered, and which is still working in the user's organism. The 10B also expresses the amount of insulin present in the user's body, but not yet used.

The calculator 26 also comprises a correction bolus module 28, a module 29 for calculating a meal bolus and a module 30 for calculating meal nutriments. The calculator 26 calculates the nutriments consumed and provides bolus or bolus modification recommendations.

A database 36 comprises a local portion 37, in the user terminal 24, that stores local data, and a remote portion 38, preferably hosted by a web server, that stores a complete replication of the data of the local portion 37. The local portion 37 of the database 36 stores, in particular, data on the foods consumed by the user, the history of consumptions, especially during meals, exercises practised by the user, amounts of insulin delivered, physical conditions of the user, and operating parameter values of the device 20.

Use algorithms 39, in communication with the database 36 and with the user terminal 24, comprise algorithms 40 for the natural language recognition of the user's voice, and algorithms 41 for analysing the user's eating patterns. The amounts of nutriments are calculated based on the nutritional composition of the foods consumed and the amounts consumed.

Adjustment algorithms 31, in communication with the database 36, comprise an algorithm 32 for detecting time events (“TimePoints”), which analyses the inputs received by the terminal 24 in relation to the time, and associates labels to them, e.g. “fasting”, “pre-prandial”, and/or “post-prandial”. The adjustment algorithms 31 also comprise an algorithm 33 for calculating the glucose ratio, or ICR, an algorithm 34 for calculating the basal insulin (slow insulin), and an algorithm 35 for calculating correction factors.

The aim of this new device is to help patients in establishing the functional insulin therapy. This consists of helping the patient with different aspects that the current tools do not provide:

A/ The importance of adjusting the values of the parameters. A large number of patients are unable to adjust their parameters themselves, or wait for a consultation to revise their functional insulin therapy parameters. These parameters are specific to each patient, and allow the insulin therapy to be tailored as closely as possible to the patient's functional needs. These parameters change over time and seasonal variations, and are strongly influenced by some events such as illness or the menstrual cycle. This change emphasises the importance of readjusting them and not waiting until the doctor's consultation for optimum blood glucose control. These parameters are:
The basal insulin. This corresponds to the minimum insulin necessary to live, and finding the right daily dose is essential for a controlled insulin therapy.
The glucose ratio corresponds to the amount of insulin needed for each gramme of carbohydrates ingested. This value varies throughout the day, and is essential for accurately calculating the dose of fast-acting insulin at meal times.
The correction factor corresponds to how much each unit of insulin reduces the blood glucose level. This value makes it possible to calculate the correction doses of fast-acting insulin, taken when the person is hyperglycaemic, or as an addition for meals.

B/ Correctly determining the amounts of nutriments in his meals. Studies show that an error of +/−10% in the amount of carbohydrates increases the risks of hypoglycaemias and hyperglycaemias after meals. A study has shown that helping, by means of an application, to calculate carbohydrates, and then the meal insulin dose, reduced the number of meal boluses that were poorly assessed and followed by a corrective action. The tools available to patients require the patient to make this assessment himself and enter an amount of carbohydrates, without providing the means for helping the patient in this task and making it easy and certain. In addition, these decision support tools do not take the impact of lipids and proteins into account.

C/ Determining the right calculation parameters and, as a result, the right insulin doses to be injected during periods of illness and during the menstrual cycle.

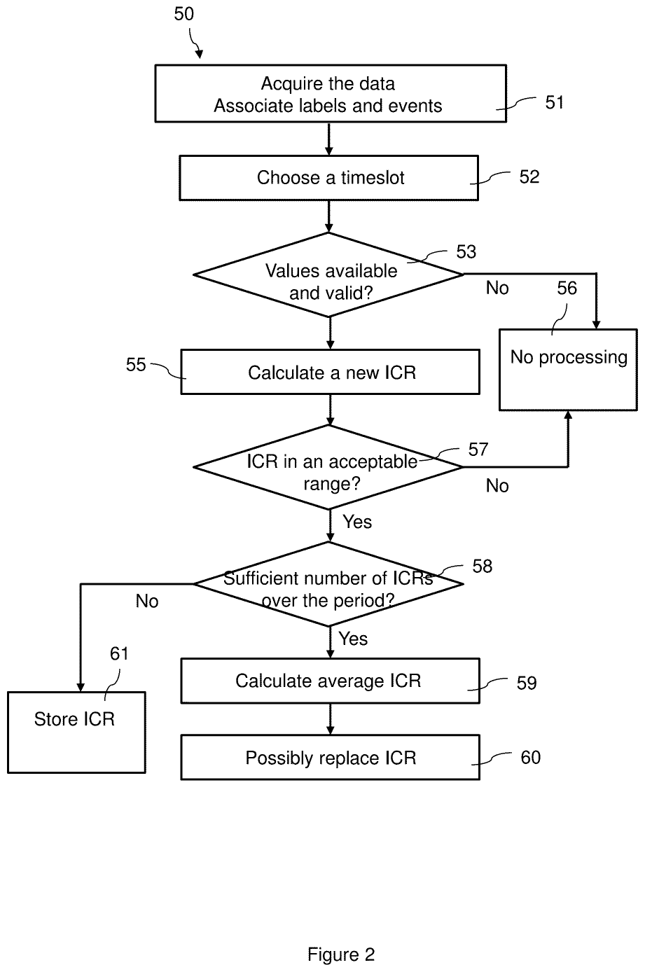

Operational algorithms of the device that is the subject of the invention, shown in the form of logic diagrams in FIGS. 2 to 10, are described below. The various methods described with reference to the figures take place at the same time.

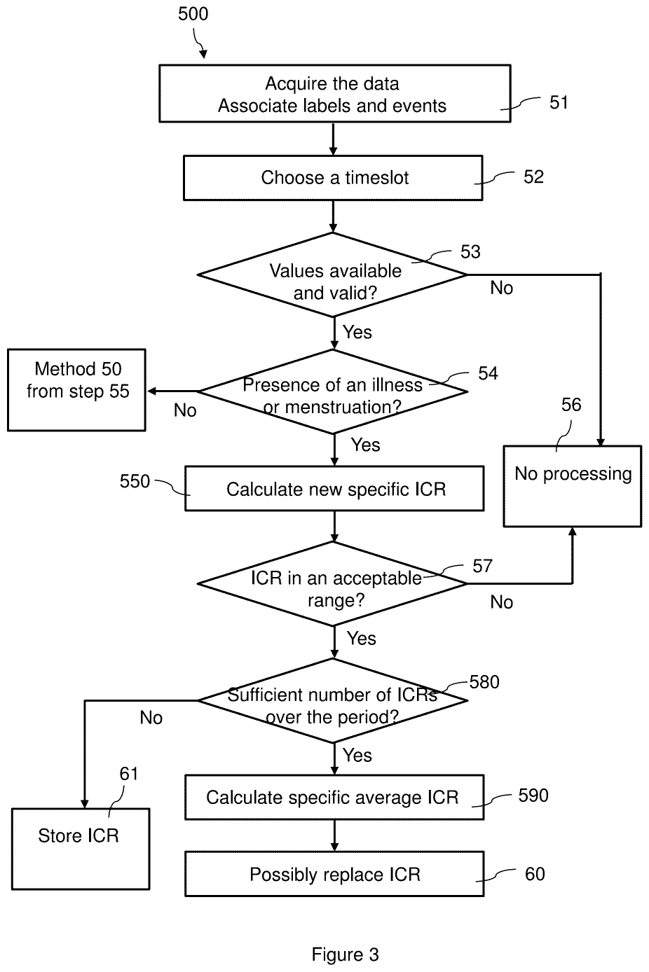

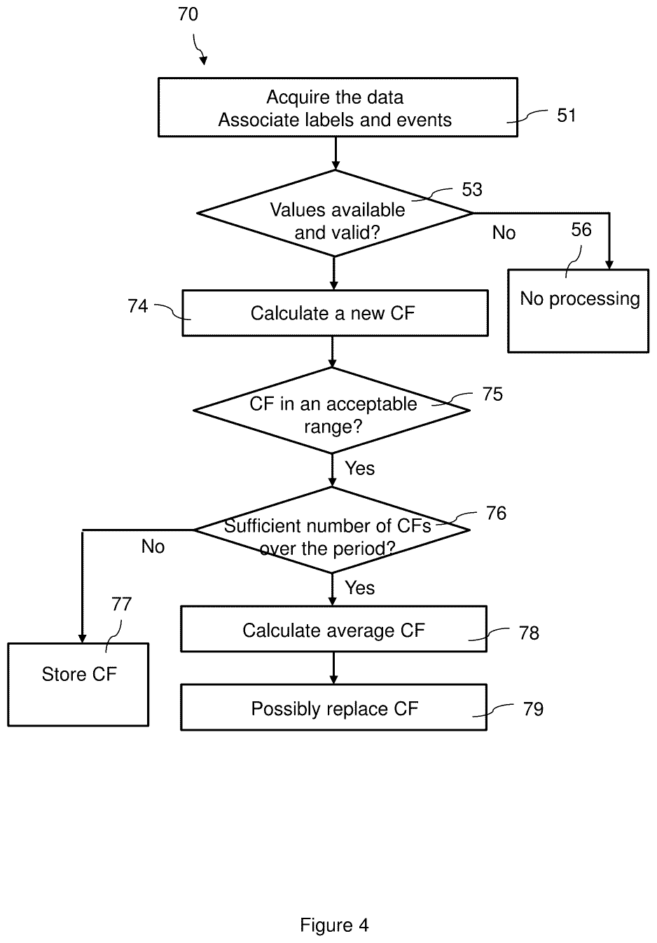

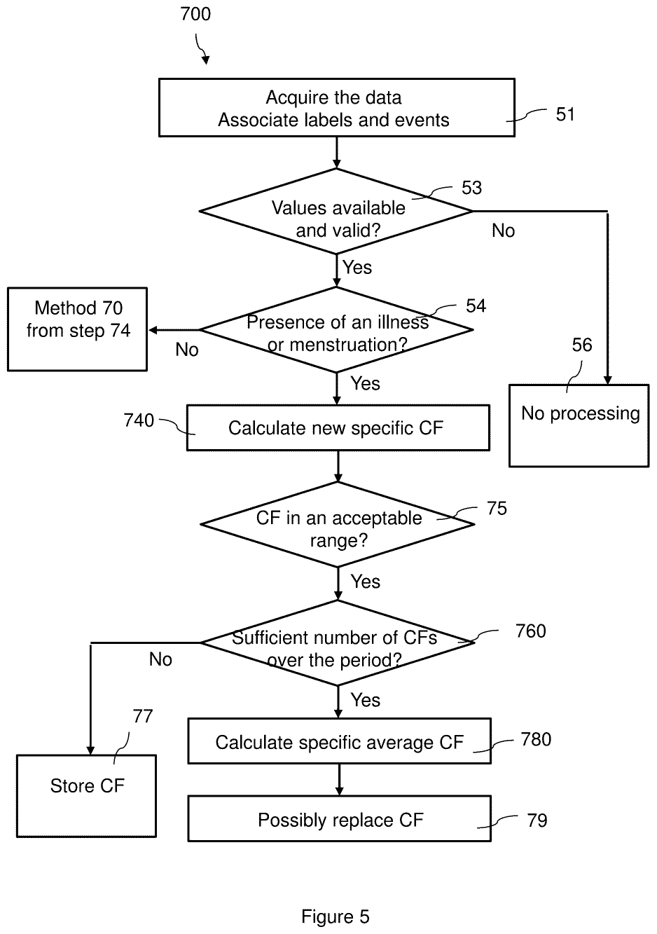

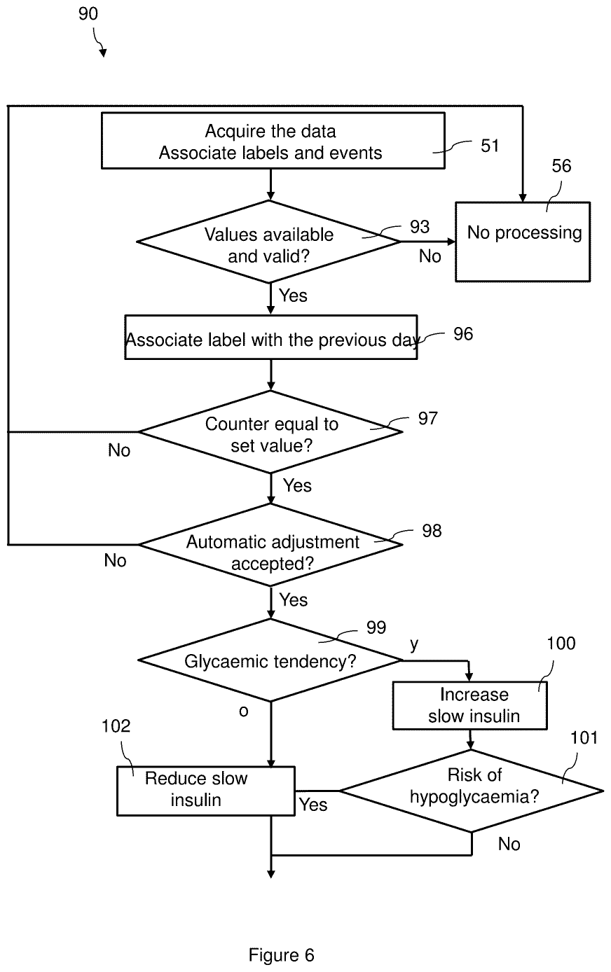

The algorithms for calculating the ICR (FIGS. 2 and 3), CF (FIGS. 3 and 4) and slow insulin dose (FIGS. 5 and 6) depend solely on the algorithm for detecting timepoints. It is noted that, usually, during use of a continuous monitor, no blood glucose value is identified. For a capillary blood glucose monitor, the patient carries out himself a limited number of blood glucose measurements at key times in the day, and assigns these measurements to key times himself.

The Timepoints algorithm used in an embodiment of the present invention makes it possible to rectify these errors in non-automatic, i.e. manually made, labelling so that the acquired data can be exploited. In particular, the Timepoints algorithm automatically assigns these data to times during a day. Such times correspond, for example, to a time for going to bed, a fasting state or a time for getting up.

With regard to such a timepoints detection algorithm, the following definitions are noted.

For type 1 blood glucose:
Pre-/Post-Prandial: Blood glucose measurements before and after a prandial injection;
Pre-/Post-Correction: Blood glucose measurements before and after a correction injection;
Pre-/Post-Manual: Blood glucose measurements before and after a manual injection;
The injections “Iₙ”, separated by a length of time “Tₙ” (Tₙ being the length of time that separates injection Iₙ and injection Iₙ₊₁).

For each injection in the day:
If Tₙ₋₁ is between two hours and five hours, Post-Iₙ₋₁=Pre Iₙ;
If Tₙ₋₁ is greater than five hours, Post-Iₙ₋₁=min(value between 2 hrs. and 5 hrs. of Iₙ₋₁);
If Tₙ₋₁ is less than two hours, Post-Iₙ₋₁=Post-Iₙ;
If Iₙ₋₁ and Iₙ are two prandial injections, they are considered as one input
and if Tₙ is less than two hours, the injections are rejected;
If Tₙ is a manual injection, and Tₙ₋₁ and Tₙ are less than two hours, the injections are rejected;
If Iₙ concerns a physical activity, injection Iₙ is rejected.

For type 2 blood glucose:
Low blood glucose (“Low Glycaemia”): Blood glucose measurement of less than 70 mg/dL;
Last Glycaemia: Blood glucose measurement collected at the end of the day; and
Fasting Glycaemia: Blood glucose measurement collected after a period of fasting (generally in the morning just after the patient gets up).

There is only one fasting glycaemia measurement and one last glycaemia measurement each day.

The maximum length of time between a fasting glycaemia measurement and a last glycaemia measurement is 12 hours. The minimum length of time between a fasting glycaemia measurement and a last glycaemia measurement is four hours.

### To Break a Fast:

By default, the period of fasting ends at noon at the latest.

But the occurrence of one of these events:
a low blood glucose level requiring a sugar dose to be taken (low glycaemia),
the start of a physical activity, or
taking an injection (with or without a meal),
identifies the end of the period of fasting (and therefore of the fasting glycaemia) as occurring just before the start of this event.

The physical activity is either entered by the patient, by means of the user interface, or detected by the device 20, by means of the connected watch 22.

### Injection:

Low Glycaemia (sugar dose),

$$Last glycaemia = last post - injection of the day ,$$

$$Fasting = first pre - glycaemia of the day ,$$

Or low glycaemia during the night.

Each of the algorithms for calculating the ICR, shown in FIGS. 2 and 3, CF, shown in FIGS. 4 and 5, and slow insulin doses, shown in FIGS. 5 and 6, requires different timepoints, but it is more efficient, economic and accurate to perform a timepoints algorithm that looks through the whole of the previous day and identifies the right labels based on events. However, in the formula, the most recent valid value of the correction factor CF is used for calculating the new glucose ratio ICR.

In some embodiments, the method for dynamically determining a slow insulin dose to be injected for a user treated by multiple injections comprises:
a step of acquiring multiple blood glucose levels of the user during a first day and the day following the first day;
a step of labelling each acquired blood glucose level according to the time of the first day and the day following the first day;
a step of determining a blood glucose level assigned to going to bed for the first day;
a step of determining a blood glucose level assigned to getting up for the day following the first day;
a step of calculating the difference in blood glucose levels between the blood glucose level for this going to bed and the blood glucose level for this getting up;
a step of determining if a predetermined event has occurred between this going to bed and this getting up;
a step of determining if a predetermined event has occurred between this going to bed and this getting up, from amongst:
having a meal between going to bed and getting up;
carrying out a physical activity between going to bed and getting up;
taking insulin between going to bed and getting up; and
possibly, an episode of hypoglycaemia.

FIG. 2 shows a method 50 for calculating the glucose ratio, or ICR. The glucose ratio ICR corresponds to how many carbohydrates have been consumed by the user.

During a step 51, the inputs for the previous day are analysed by utilising an algorithm for detecting time events (“TimePoint”). Step 51 consists of associating labels to events amongst, for example, “pre-prandial”, “post-prandial”, “fasting”, “getting up” and “going to bed”.

During step 51, during the acquisition of data, for example from capillary blood glucose monitors, continuous glucose monitors and connected pens, these data are processed to associate them, for example, to data relating to meals provided by the user. The same applies to the insulin values calculated and proposed to the user. For example, during step 51, the following steps are implemented: The data is received from connected medical devices.

The information is recorded in the database depending on the nature of the data received.

The data is analysed by different algorithms in order to label, in other words assign a label to, some inputs based on other input events, to make better use of the blood glucose levels and insulin. Through, for example:
detecting pre-prandial blood glucose levels and post-prandial blood glucose levels;
detecting the fasting blood glucose level, and the blood glucose level on going to bed; and/or
associating insulin doses coming from the connected pen with the insulin doses suggested.

This processing avoids false duplicates. For example, in the situation where the patient makes a calculation and delays making his injection, the above method makes it possible to avoid duplication between inputs, and to associate the calculated dose with the actual time of the injection.

During a step 52, a specific interval of time (“timeslot”) is chosen, for example a meal timeslot, between breakfast, lunch, afternoon snack and dinner, dinner and breakfast of the following day, going to bed and breakfast of the following day.

During a step 53, it is determined whether all the necessary values are available and valid. For example, these comprise the following values:
a pre-prandial blood glucose value;
a post-prandial blood glucose value;
an amount of carbohydrates;
the description of a meal; and
the active insulin, also called 10B, at the time of the bolus injection.

If not, the timeslot processing is rejected, during a step 56. If yes then, during a step 55, a new glucose ratio ICR, newlCR, is calculated as a function of the following factors:
pre-prandial blood glucose level;
post-prandial blood glucose level;
amount of carbohydrates;
bolus;
active insulin;
CF value;
upper target blood glucose level; and
lower target blood glucose level.

During a step 57, it is determined whether this new glucose ratio is within an acceptable range of values, since, despite the previous steps, there can sometimes be incorrect inputs (manual input error, etc.) that can give an abnormal result.

If not, the timeslot processing is rejected, during step 56. If yes then, during a step 58, it is determined whether there is a sufficient number of new glucose ratios over a predefined period of time. For example, 7 new glucose ratios during the last 30 days. If not then, during a step 61, the timestamped new glucose ratio is stored, and one waits for the following day (processed as described in step 51). If yes then, during a step 59, a new average glucose ratio is calculated, equal to the average of the glucose ratios during the predefined period of time. This is a weighted average taking into account the average of these 7 new glucose ratios, and also the old glucose ratio used.

Then, during a step 60, if the new average glucose ratio is within a range of values around the old glucose ratio (for example, between 90% and 110% of the old ICR value, oldICR), the glucose ratio to be applied for the user is not changed and, if the new glucose ratio is outside this range, the old glucose ratio is replaced by the new average glucose ratio. It is therefore the new glucose ratio ICR that is applied for the user.

FIG. 3 shows a method 500 for calculating the specific glucose ratio, or specific ICR. Such a specific ICR is calculated when the presence of an illness, menstruation or physical activity is determined. It is noted that the method 500 is implemented optionally with regard to method 50.

In particular, during a step 54, the presence of an illness or menstrual cycle is determined by asking the user. If yes, a new specific intermediate ICR is calculated during step 550 as a function of factors similar to those mentioned for step 55. It is noted that such factors are specific to the user's particular state, i.e. to the presence of an illness, menstruation, or physical activity. The following steps are similar to those mentioned for method 50. In particular, the step 580 of method 500 differs from the step 58 of method 50 since another period is taken into consideration. The period, and therefore the sufficient number of new specific ICRs, for step 580 will be less than the period associated to step 58 since the presence of a specific event is considered to be a one-off event. In addition, the step 590 of method 500 differs from the step 59 of method 50 since a new specific average ICR is calculated, not a so-called “standard” ICR obtained during the implementation of method 50, and especially following step 59, the standard ICR being calculated when no particular state is determined. FIG. 3 shows that if no particular state is determined, the so-called “standard” method 50 is carried out.

Therefore, in some embodiments, such as the one shown in method 500, if an event such as the presence of an illness or menstruation is determined, specific processing is applied taking a specific ICR into consideration. In particular, such processing determines and adjusts the specific parameters associated to such an event. Therefore, this specific parameter can be used during the following calculations with the same characteristics, as described in the steps 119, 134 or 144 mentioned in the rest of the description.

FIG. 4 shows a method 70 for calculating a correction factor CF, performed once a day. The initial steps, 51, 53 and 56, have already been detailed with regard to FIG. 2.

During a step 74, a new correction factor CF for the insulin-sensitivity of the user is calculated. The correction factor CF corresponds to the factor lowering blood glucose levels for a unit of insulin received. The new correction factor, newCF, is a function of the following factors:
pre-correction blood glucose level;
post-correction blood glucose level;
bolus;
active insulin;
upper target blood glucose level; and
lower target blood glucose level.

During a step 75, it is determined whether the new value of the correction factor CF is within an acceptable range of values (for example, between 0 and 2 times oldCF).

If not, the method terminates. If yes then, during a step 76, it is determined whether there is a sufficient number of new correction factors CF over a predefined period of time. For example, 7 new correction factors during the last 30 days. If not, during a step 77, the timestamped new correction factor CF is stored, and one waits for the following day (processed as described in step 51). If yes then, during a step 78, a new average correction factor is calculated, equal to the average of the correction factors CF during the predefined period of time. Then, during a step 79, if the new average correction factor is within a range of values around the old correction factor (for example, between 90% and 110% of the old value), the correction factor CF to be applied for the user is not changed and, if the new correction factor is outside this range, the old correction factor is replaced by the new average correction factor.

FIG. 5 shows a method 700 for calculating a specific correction factor CF. Such a specific CF is calculated when a presence of an illness, menstruation or physical activity is determined. It is noted that the method 700 is implemented optionally with regard to method 70.

In particular, during a step 54, the presence of an illness or menstrual cycle is determined by asking the user. If yes, a new specific intermediate CF is calculated during step 740 as a function of factors similar to those mentioned for step 74. It is noted that such factors are specific to the user's particular state, i.e. to the presence of an illness, menstruation, or physical activity. The following steps are similar to those mentioned for method 700. In particular, the step 760 of method 700 differs from the step 76 of method 70 since another period is taken into consideration. The period, and therefore the sufficient number of new specific CFs, for step 760 will be less than the period associated to step 76 since the presence of a specific event is considered to be a one-off event. In addition, the step 780 of method 700 differs from the step 78 of method 70 since a new specific average CF is calculated, not a so-called “standard” CF obtained during the implementation of method 70, and especially following step 78, the standard CF being calculated when no particular state is determined. FIG. 5 shows that if no particular state is determined, the so-called “standard” method 70 is carried out.

Therefore, in some embodiments, such as the one shown in method 700, if an event such as the presence of an illness or menstruation is determined, specific processing is applied taking a specific CF into consideration. In particular, such processing determines and adjusts the specific parameters associated to such an event. Therefore, this specific parameter can be used during the following calculations with the same characteristics, as described in the steps 119, 134 or 144 mentioned in the rest of the description.

FIG. 6 shows a method 90 for calculating the basal insulin. The initial steps, 51 and 56, have already been detailed with regard to FIG. 2.

During step 93, the availability and validity of the values are verified. If the values are available, their validity is checked, especially if a predetermined event has occurred between this going to bed and this getting up or this fasting state. Such an event corresponds, for example, to:
having a meal between going to bed and getting up, or fasting state;
carrying out a physical activity between going to bed and getting up, or the fasting state;
taking insulin between going to bed and getting up, or the fasting state; and/or
a hyperglycaemia or hypoglycaemia.

If a predetermined event has occurred, the associated timeslot is considered invalid. There is consequently no processing for this timeslot, corresponding to step 56.

If the timeslot is validated, step 96 is carried out.

During a step 96, a label vector is associated to the last day. Taking the previous day into account, in a similar way to step 93, is a function of the getting up or fasting blood glucose level for the current day and of the going to bed blood glucose level for the previous day, and possibly of events that occurred between going to bed and getting up, for example night-time meal, night-time hypoglycaemia or hyperglycaemia, taking insulin, carrying out a physical activity during the night.

This analysis makes it possible to give:
a “normal” label, if the analysis shows that the period of night-time fasting occurred correctly, indicating that the basal insulin dose was correct;
a “hyper” label, if the analysis shows that the slow insulin dose was low and led to an increase in the blood glucose level of more than 20 mgl/dl, for example; and
a “hypo” label, if the analysis shows that the slow insulin dose was high and led to a fall in the blood glucose level of more than 20 mg/dl, for example.

During a step 97, it is determined whether a counter is equal to a predefined value, for example the predefined value utilised during steps 58 and 76. In addition, the counter is associated to a particular number of days corresponding to a first predefined period, for example 7 days. In other words, the counter makes a count over a period of seven days.

If the counter is not equal to such a predefined value, the slow insulin value is not changed, and one waits for the following day. If it is then, during a step 98, it is determined whether a decision has been taken (and stored as an operating parameter value of the device) to make an automatic adjustment. If not, the basal insulin value is not changed, and one waits for the following day. If yes then, during a step 99, it is determined whether there is a tendency to hypoglycaemia, output “o”, or to hyperglycaemia, output “y”. In particular, a tendency to hypoglycaemia is indicated when the number of hypoglycaemias determined is greater than or equal to 2 over a first predefined period of 7 days. Similarly, a tendency to hyperglycaemia is indicated when the number of hyperglycaemias determined is greater than or equal to 2 over a first predefined period of 7 days.

In the case of a tendency to hyperglycaemia then, during a step 100, the slow insulin is increased by one unit and the counter whose value is compared to the predefined value during the step 97 is reset to zero. The counter is increased in a case where the result of the step 97 is “No”. When this counter has reached the value of seven, as long as the basal insulin value (output “yes” from step 98) is not changed, it remains at this value of seven.

Then, optionally, during a step 101, the label vector is checked for the next two days: If just one “hypo” label occurs in the next two days, which represents a fall in the blood glucose level of more than 20 mg/dl between going to bed and getting up, then there is a risk of hypoglycaemia. Therefore, in step 102, the insulin is reduced by one unit and the counter is reset. If not, the method is ended for the current day. If yes then, during a step 102, the basal insulin is reduced by one unit and the counter whose value is compared to the predefined value during the step 97 is reset to zero, and the method is ended for the current day. If the output from step 99 is “o”, step 102 is carried out and the method is ended for the current day.

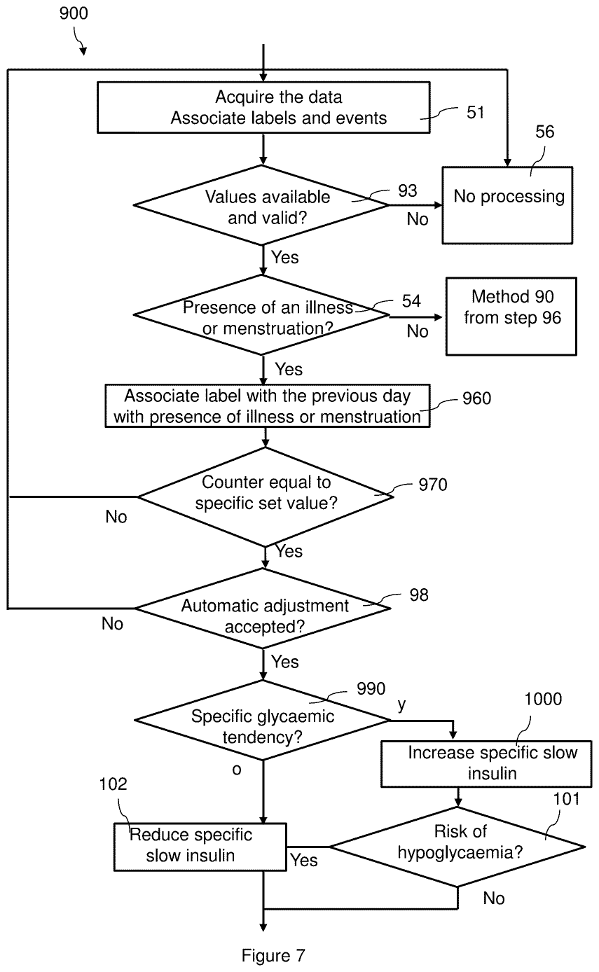

FIG. 7 shows a method 900 for calculating the specific basal insulin. Such a specific insulin is calculated when a presence of an illness, menstruation or physical activity is determined. It is noted that the method 900 is implemented optionally with regard to method 90. The initial steps, 51, 56 and 93, have already been detailed with regard to FIG. 6.

Before the step 93, and if all the data are available and valid, one proceeds to step 54. During a step 54, the presence of an illness or menstrual cycle is determined by asking the user. If yes, a label association with the previous day is carried out in step 960, where the previous day must be a day that is equally specific, i.e. with the presence of an illness, menstruation. In other words, for cases of illness or menstruation, one uses the data for days having the same specific characteristic corresponding to the illness or the presence of menstruation.

During a step 970, it is determined whether a counter is equal to a predefined value, for example the predefined specific value utilised during steps 580 and 760. In addition, the counter is associated to a particular number of days corresponding to a first predefined period specific to the event (illness or menstruation). For example, such a period is equal to 3 days. In other words, the counter makes a count over a period of 3 days.

If the counter is not equal to such a predefined value, the slow insulin value is not changed, and one waits for the following day. If it is then, during a step 98, it is determined whether a decision has been taken (and stored as an operating parameter value of the device) to make an automatic adjustment. If not, the basal insulin value is not changed, and one waits for the following day. If yes then, during a step 990, it is determined whether there is a tendency to specific hypoglycaemia, output “o”, or to specific hyperglycaemia, output “y”. In particular, a tendency to specific hypoglycaemia is indicated when the number of hypoglycaemias determined is greater than or equal to 1 over a first predefined period of 3 days. Similarly, a tendency to specific hyperglycaemia is indicated when the number of hyperglycaemias determined is greater than or equal to 1 over a first predefined period of 3 days.

In the case of a tendency to hyperglycaemia then, during a step 1000, the slow insulin is increased, for example, by one unit and the counter whose value is compared to the predefined value during the step 970 is reset to zero. The counter is increased in a case where the result of the step 970 is “No”. When this counter has reached the value of three, as long as the basal insulin value (output “yes” from step 98) is not changed, it remains at this value of three.

The following steps are similar to those described above for method 90.

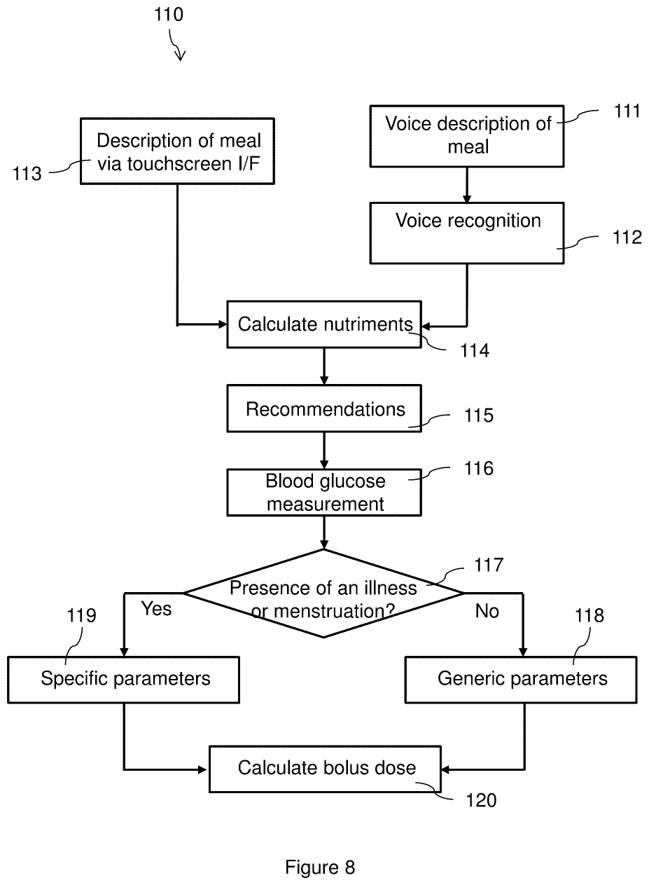

FIG. 8 shows a method 110 for calculating a meal bolus. Depending on the use of the user interface by the user, then either step 113 (manual input), or the steps 111 and 112 (input by speech recognition), are carried out. In both cases, the user answers a questionnaire to describe the foods in his meal. In the case of voice dictation (step 111), natural language speech recognition is utilised during step 112.

During a step 114, following step 112 or step 113, the nutriments corresponding to the meal, i.e. the number of calories, carbohydrates, lipids and proteins, are calculated.

During a step 115, recommendations regarding the meal are given, utilising an algorithm for analysing diet patterns. For example, each input concerns an item of data:
pre-prandial blood glucose level;
post-prandial blood glucose level;
bolus;
food 1 and amount 1;
food 2 and amount 2; etc.

For each food to be analysed: one searches for all occurrences of food 1 in the data contained in the history.

If there is a correlation between the presence of these foods and a tendency to hypoglycaemia or hyperglycaemia after meals, then this warning will be triggered.

The correlation can be determined through a data processing algorithm, such as linear regression, logistic regression, k-nearest neighbour, support vector machine, random forest.

For example, a recommendation warns a user about a food included in the meal, for which the algorithm has observed a tendency to lead to a hypoglycaemia in this user.

During a step 116, a measurement of the glucose level in the user's blood over a certain length of time, for example the fifteen minutes before the start of the meal, is obtained from at least one connected device. During a step 117, the presence of an illness or menstrual cycle is determined by asking the user. If not then, during a step 118, generic calculation parameters are obtained. The parameters are the ICR and CF and the upper and lower target blood glucose levels. The generic, or known as standard, glucose ratio ICR and correction factor CF are those which are adjusted by the algorithms described above for FIGS. 2, 4 and 6. The specific Timepoints algorithm calculation parameters are determined by using an adaptation of the three algorithms above, but using some different parameters (for the number of days, three instead of seven for example, the method for calculating the weighted averages), and only taking into account the values concerned by the same state, as described above for FIGS. 3, 5 and 7.

Therefore, in the Cloud and local database, there is a generic glucose ratio ICR, a glucose ratio for an illness “ICRᵢₗₗₙₑₛₛ”, a glucose ratio for menstruation “ICRₘₑₙₛₜᵣᵤₐₜᵢₒₙ”, etc. And similarly for the specific correction factor CF and the specific basal insulin dose. If yes then, during a step 119, calculation parameters specific to the illness or menstrual cycle are obtained.

Following one of steps 118 or 119, during a step 120, the meal bolus dose is calculated, utilising the calculation parameters obtained, as a function of the values of the glucose ratio ICR and correction factor CF parameters.

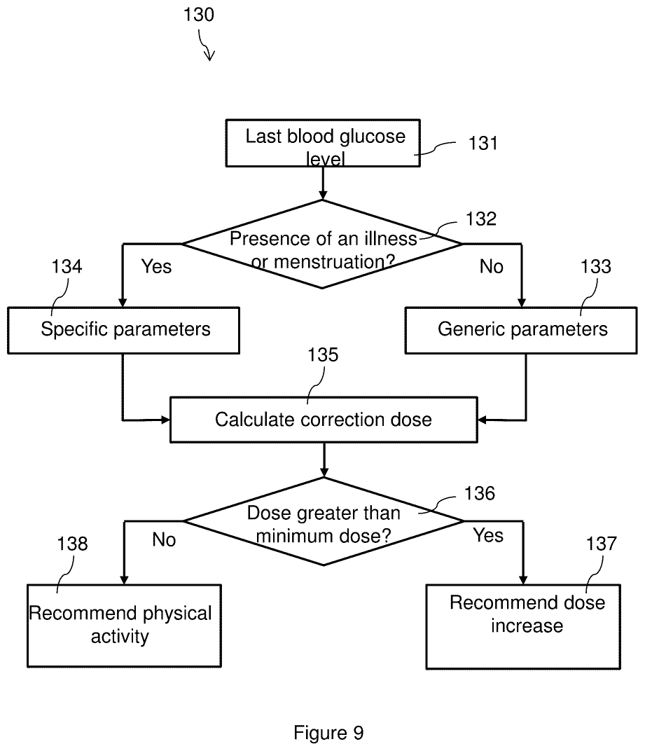

FIG. 9 shows a method 130 for correcting the bolus. During a step 131, the last blood glucose level measured in the user's blood is obtained. During a step 132, the presence of an illness or menstrual cycle is determined by asking the user. If not then, during a step 133, generic calculation parameters are obtained, as described above. If the result of step 132 is positive then, during a step 134, calculation parameters specific to the illness or menstrual cycle, such as “ICRᵢₗₗₙₑₛₛ”, “ICRₘₑₙₛₜᵣᵤₐₜᵢₒₙ”, “CFᵢₗₗₙₑₛₛ” and “CFₘₑₙₛₜᵣᵤₐₜᵢₒₙ” are obtained, as described above.

Following one of steps 133 or 134, during a step 135, a correction insulin dose is calculated. For example, a Correction Bolus type of formula is used based on the blood glucose level, correction factor, CF, upper target blood glucose level and lower target blood glucose level, utilising the calculation parameters obtained. During a step 136, it is determined whether the correction insulin dose calculated is greater than or equal to a minimum injectable insulin dose. If yes then, during a step 137, an increase in the fast-acting insulin dose injected is recommended to the user through the user interface. If not then, during a step 138, a physical activity making it possible to obtain the same result as the correction insulin dose is recommended to the user through the user interface.

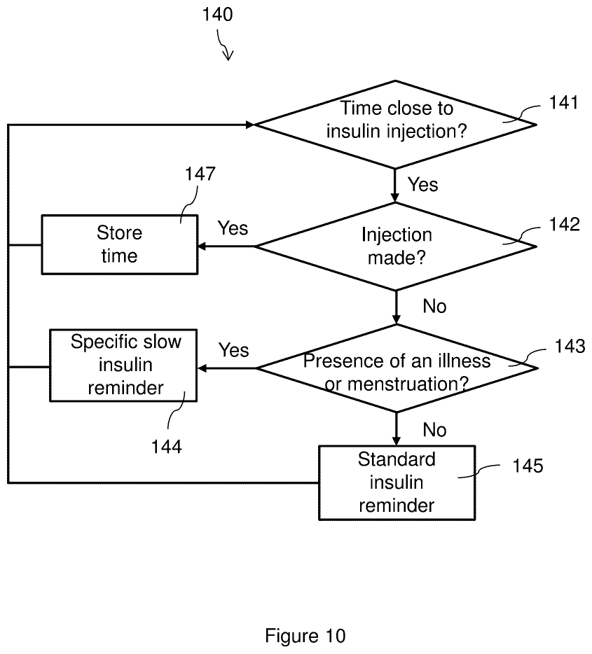

FIG. 10 shows a method 140 for determining the slow insulin. During a step 141, it is determined whether the current time is close to the insulin injection time, for example less than 15 or 30 minutes from such a time stored in memory. If not, the method ends. If yes, during a step 142, it is determined whether an injection is made, by means of a connected pen or a declaration by the user utilising the user interface. If yes, during a step 147, the time of this injection, the dose injected, the foods ingested, the blood glucose level and the operating parameter values of the device are stored, and the method ends. If not, during a step 143, the presence of an illness or menstrual cycle is determined by asking the user. If not then, during a step 145, the user is reminded of the basal insulin dose to be injected, and one returns to step 141. If yes then, during a step 144, the user is reminded of the event-specific basal insulin dose to be injected, and one returns to step 141. The specific slow insulin dose is the value adjusted during the utilisation of the algorithm from FIG. 7.

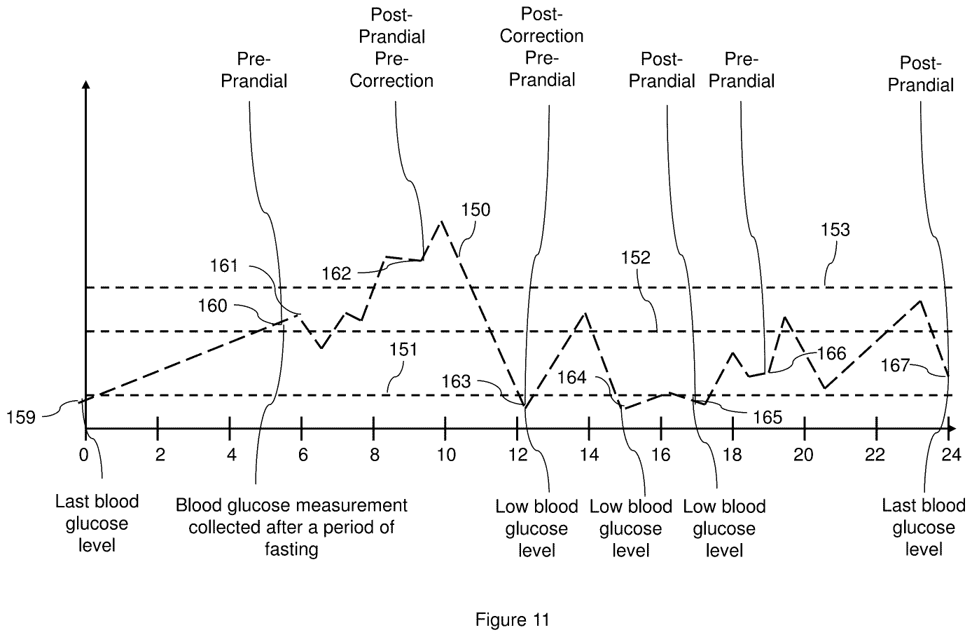

FIG. 11 shows, by way of illustration, a schematic curve 150 of the blood glucose level of a patient during a typical day, the times being indicated on the x-axis. FIG. 8 also shows the low blood glucose (less than 70 mg/dl) 151, high blood glucose (greater than 140 mg/dl) 152 and very high blood glucose (greater than 180 mg/dl) 153 levels.

At an instant 159 in the previous day, the timepoints algorithm 32 identifies, amongst the blood glucose levels, the last glucose level of the day, last glycaemia, which is therefore identified and stored. The device's memory therefore contains the previous day's timestamped data and the values of the glucose ratio ICRn, ICRavg, CFn, CFavg and IB for each timeslot.

At an instant 160 early in the morning, the algorithm 32 detects a pre-prandial measurement, which corresponds to the fasting glycaemia, a blood glucose measurement acquired after a period of fasting of at least four hours, because no event occurred during the night.

At an instant 161 before eating the first meal of the day, the user describes the envisaged first meal. The nutriments of this first meal are therefore determined, then meal recommendations, as described above, and insulin recommendations, as described above for the bolus calculation, are calculated. These recommendations are given to the user through the user interface. The user then takes the insulin and eats the first meal.

At an instant 162 in the morning, the user makes a post-prandial, pre-correction blood glucose measurement. The device therefore calculates a possible correction bolus, as described above, and transmits it to the user through the user interface.

At an instant 163 before eating the second meal of the day, the user makes a blood glucose measurement and describes the envisaged second meal. The nutriments of this second meal are therefore determined, then meal recommendations, as described above, and insulin recommendations, as described above for the bolus calculation, are calculated. These recommendations are given to the user through the user interface. The user then takes the insulin and eats the second meal.

At instant 164, the patient has a low blood glucose level. The application reacts by recommending taking a sugar dose, and checking his blood glucose level 15 or 30 minutes later. The user can possibly parametrise his preferred sugar doses (his preferred sugary foods), for greater accuracy, and the application gives him the number of such sugar doses to take.

At an instant 165, the user makes a post-prandial blood glucose measurement. The device, possibly, calculates a correction bolus, as described above, and transmits a recommendation to the user through the user interface. However, in FIG. 11, instant 165 also corresponds to a low blood glucose level. Therefore, the device recommends a sugar dose, as described above, not a correction bolus.

At an instant 166 before eating the third meal of the day, the user makes a blood glucose measurement and describes the envisaged third meal. The nutriments of this third meal are therefore determined, then meal recommendations, as described above, and insulin recommendations, as described above for the bolus calculation, are calculated. These recommendations are given to the user through the user interface. The user then takes the insulin and eats the third meal.

At instant 167, for example just before he goes to bed, the user makes a post-prandial blood glucose measurement. The device, possibly, calculates a correction bolus, as described above, and transmits a recommendation to the user through the user interface.

At a predefined time in the day, not shown in FIG. 11, the device associates labels to the instants of the previous day, and calculates new values of the glucose ratio in the morning, ICR_{morning}, afternoon, ICR_{afternoon}, evening, ICR_{evening} and night, ICR_{night}, the correction factor CF and the basal insulin.

Preferably, the means of the device 20 are configured to implement the steps of the methods 50, 500, 70, 700, 90 and 900 and their embodiments as described above, and the method 90 and its different embodiments can be implemented by the means of the device 50, 500, 70, 700, 90 and 900.

1. A Device for dynamically determining a slow insulin dose to be injected for a user treated by multiple injections, comprising:
a means for automatically acquiring multiple blood glucose levels during a first day and the day following the first day;
a means for automatically labelling each acquired blood glucose level according to the time of the first day and the day following the first day;
a means for determining a blood glucose level assigned to going to bed for the first day;
a means for determining a blood glucose level assigned to a fasting state or getting up for the day following the first day;
a means for calculating the difference in blood glucose levels between the blood glucose level for this going to bed and the blood glucose level for this getting up or fasting state;
a means for determining if a predetermined event has occurred between this going to bed and this getting up or fasting state;
a means for determining, if no predetermined event has occurred, a hyperglycaemia or a hypoglycaemia depending on the difference in blood glucose levels;
a means for determining if at least a predetermined number of hyperglycaemias or hypoglycaemias has occurred in a predetermined period of time;
a means for modifying the slow insulin dose to be injected if at least the predetermined number of hypoglycaemias or hyperglycaemias has occurred in the first predetermined period of time; and
a means for indicating to the user a numerical value of the modified slow insulin dose to be injected.

2. The device according to claim 1, which also comprises:
a means for detecting an increase in the numerical value of the modified slow insulin dose to be injected;
a determination means, activated by the detection means, for determining whether at least a predetermined number of hypoglycaemias greater than or equal to one has occurred in a second predetermined period of time, the second period of time being less than the first period of time; and
a correction means for correcting the numerical value of the slow insulin dose to be injected if at least the predetermined number of hypoglycaemias has occurred in the second period of time, the correction means being configured to transmit to the indication means a corrected numerical value of the slow insulin dose to be injected.

3. The device according to claim 1, wherein the means for determining if a predetermined event has occurred between this going to bed and this getting up is configured to determine the occurrence of at least one event from amongst:
having a meal between going to bed and getting up, or fasting state;
carrying out a physical activity between going to bed and getting up, or fasting state; and
taking insulin between going to bed and getting up, or fasting state.

4. The device according to claim 1, which also comprises:
a means for determining the occurrence of an event from amongst an illness of the user, the user's menstruation, and carrying out a physical activity;
a means for determining whether at least a predetermined number, greater than or equal to one, of hypoglycaemias or hyperglycaemias has occurred in a predetermined period of time specific to this event, the means for determining the number of hypoglycaemias or hyperglycaemias being activated by the occurrence of a said event; and
a means for modifying the slow insulin dose to be injected by a specific slow insulin dose, if at least the predetermined number of hypoglycaemias or hyperglycaemias has occurred in the predetermined specific period of time, the indication means indicating to the user a numerical value of the specific slow insulin dose to be injected if the occurrence of a said event is determined.

5. The device according to claim 4, which also comprises a means for determining a correction bolus or a meal bolus, based on:
the last blood glucose level measured in the user's blood;
a correction factor;
an upper target blood glucose level; and
a lower target blood glucose level,
if the occurrence of an event, from amongst an illness of the user, the user's menstruation, and carrying out a physical activity, is determined.

6. The device according to claim 5, which comprises a means for determining whether the correction bolus is higher than a minimum injectable insulin dose; and:
if it is, transmitting to the user the value of the correction bolus to be injected; and
if not, transmitting to the user a value representative of a physical activity making it possible to obtain the same effect as the corrective insulin dose.

7. The device according to claim 1, which also comprises:
a means for determining a blood glucose level of the user before a meal, called the “pre-prandial blood glucose”, the pre-prandial blood glucose being labelled by the assignment means;
a means for determining a blood glucose level of the user after a meal, called the “post-prandial blood glucose”, the post-prandial blood glucose being labelled by the assignment means;
a means for receiving a description of a meal;
a means for determining an insulin active at the time of the injection;
a means for determining a new glucose ratio (ICR) for a meal timeslot between the acquisition instants labelled as pre-prandial blood glucose level and those labelled as post-prandial blood glucose level, as a function of the following factors:
a pre-prandial blood glucose level;
a post-prandial blood glucose level;
an amount of carbohydrates absorbed during the meal;
a bolus;
a level of active insulin;
a value of a correction factor;
an upper target blood glucose level; and
a lower target blood glucose level; and
a means for replacing a glucose ratio used by the device for determining a fast-acting insulin dose to be injected, representative of a number of carbohydrates absorbed by the user, if at least a predetermined number, greater than or equal to two, of new glucose ratios were determined in a predetermined period of time, the replacement glucose ratio being an average, possibly weighted, of the new glucose ratios.

8. The device according to claim 7, wherein the determining means determines a new specific glucose ratio for a meal timeslot as a function, additionally, of the following factors:
an illness of the user;
the user's menstruation; and/or
carrying out a physical activity,
the device also comprising a means for replacing a glucose ratio by a specific glucose ratio used by the device for determining a specific fast-acting insulin dose to be injected, the replacement specific glucose ratio being determined as a function of the presence of an illness and/or menstruation.

9. The device according to claim 7, wherein the means for replacing a glucose ratio is configured to inhibit the replacement of the glucose ratio if the replacement glucose ratio is within a predefined range of values around the glucose ratio to be replaced.

10. The device according to claim 7, which also comprises:
a means for calculating nutriments corresponding to the meal described, number of calories, carbohydrates, lipids and proteins;
a means for determining a correlation between the presence of the foods described and a tendency to hypoglycaemia or hyperglycaemia after meals; and
a means for triggering an alert for the user, if a sufficiently high correlation is detected; and
a means for determining a meal bolus dose, as a function of the glucose ratio and correction factor values.

11. The device according to claim 1, which also comprises a means for the daily determination of a new correction factor (CF) corresponding to a factor lowering blood glucose levels for a unit of insulin received, the correction factor being a function of the following factors:
a pre-correction blood glucose level;
a post-correction blood glucose level;
a bolus;
an active insulin;
an upper target blood glucose level; and
a lower target blood glucose level; and
a means for replacing a correction factor for determining a fast-acting insulin dose to be injected, if at least a predefined number, greater than or equal to two, of new correction factors were determined in a predetermined period of time, the replacement correction factor being an average, possibly weighted, of the new correction factors.

12. The device according to claim 11 wherein the daily determining means determines a new specific correction factor as a function, additionally, of the following factors:
an illness of the user; and/or
the user's menstruation,
the device also comprising a means for replacing a correction factor by a specific correction factor for determining a specific fast-acting insulin dose to be injected, the replacement specific correction factor being determined as a function of the presence of an illness and/or menstruation.

13. The device according to claim 11, wherein the means for replacing a correction factor is configured to inhibit the replacement of the correction factor if the replacement correction factor is within a predetermined range of values around the correction factor to be replaced.

14. A method for dynamically determining a slow insulin dose to be injected for a user treated by multiple injections, characterised in that it comprises:
a step of automatically acquiring multiple blood glucose levels during a first day and the day following the first day;
a step of automatically labelling each acquired blood glucose level according to the time of the first day and the day following the first day;
a step of determining a blood glucose level assigned to going to bed for the first day;
a step of determining a blood glucose level assigned to a fasting state or getting up for the day following the first day;
a step of calculating the difference in blood glucose levels between the blood glucose level for this going to bed and the blood glucose level for this getting up or fasting state;
a step of determining if a predetermined event has occurred between this going to bed and this getting up or fasting state;
a step of determining, if no predetermined event has occurred, a hyperglycaemia or a hypoglycaemia depending on the difference in blood glucose levels;
a step of determining if at least a predetermined number of hyperglycaemias or hypoglycaemias has occurred in a predetermined period of time;
a step of modifying the slow insulin dose to be injected if at least the predetermined number of hypoglycaemias or hyperglycaemias has occurred in the predetermined specific period of time; and
a step of indicating to the user a numerical value of the modified slow insulin dose to be injected.
